(19) United States
(12) Patent Application Publication
(10) Pub. No.: US 2007/0148262 A1
(43) Pub. Date: Jun. 28, 2007

(54) Bactericidal and virucidal composition containing natural products

(76) Inventors: Jeong Chan Ra, Suwon (KR); Yong Ho Park, Seoul (KR); Bong Kyun Park, Suwon-si (KR)

Correspondence Address: INTELLECTUAL PROPERTY / TECHNOLOGY LAW, PO BOX 14329, RESEARCH TRIANGLE PARK, NC, 27709, US

(21) Appl. No.: 11/318,871

(22) Filed: Dec. 27, 2005

(51) Int. Cl. A61K 36/752; A61K 36/736; A61K 31/19

(52) U.S. Cl. 424/735; 424/736; 514/574; 514/55

(57) ABSTRACT

A bactericidal and virucidal composition containing the natural products malic acid and grapefruit seed extract. Such compositions exhibit disinfectant effects on a broad spectrum of bacteria and viruses, and are particularly useful for disinfection of stalls, domestic animals and drinking water.

### BACKGROUND OF THE INVENTION

1. Field of the Invention

The present invention relates to a bactericidal and virucidal composition containing natural products, and more particularly, to a bactericidal and virucidal composition containing malic acid and grapefruit seed extract.

2. Background of the Related Art

Pathogens which have been of recent interest in the livestock field include avian influenza virus, foot-and-mouth disease virus, hog cholera virus, Salmonella, Streptococcus, and the like.

Avian influenza viruses, which cause much damage to poultry, such as hens and turkeys, are classified into highly pathogenic avian viruses, low pathogenic avian viruses and non-pathogenic avian viruses according to their pathogenicity. The highly pathogenic avian viruses are classified as “List A species” by the Office International des Epizootics (OIE) and as class I infectious livestock diseases in Korea. Avian influenza is caused by type A viruses, including H and N serotypes. In these two types of proteins, a total of 135 viral serotypes can be found, and 15 HA serotypes and 9 NA serotypes had been reported by 2004. Infection occurs mainly by direct contact with avian excreta and also spreads by droplets, water, human feet, feeding cars, devices, feces attached to the outer surface of eggs, and the like.

In the symptoms of the viral infection, respiratory symptoms, diarrhea and a rapid decrease in egg production are shown, although the symptoms vary depending on the pathogenicity of infected viruses. In some cases, head portions, such as crests, show cyanosis, and sometimes edema appears on the face or feathers flock together at one point. Mortality caused by the viral infection varies from 0% to 100% depending on the viral pathogenicity. The viral infection requires precise diagnosis because its symptoms are similar to those of other diseases such as Newcastle disease, infectious laryngotracheitis, mycoplasma infections, and the like.

Avian influenza outbreaks did not occur in the world after the 1930s, until comparatively recently, when they began in Europe, including Belgium and France, in 1983. By 2004, highly pathogenic avian influenza outbreaks, including low-pathogenic avian influenza, had occurred in all countries of the world. The highly pathogenic avian influenza has infected human beings and killed 6 people in Hong Kong in 1997 and 16 people in Vietnam in 2004. In Korea, avian influenza outbreaks occurred in Eumsung-koon, Chungcheongbuk-do in 1996 and again in December 2003 and spread nationwide, but they were found to be low pathogenic and non-infectious to the human body.

When avian influenza outbreaks occur, most countries respond by killing all of the infected animals involved in the outbreak, and countries experiencing outbreaks cannot export poultry products. Accordingly, avian influenza viruses can be regarded as being among primary factors that interfere with the development of the livestock industry.

For the development of the livestock industry, a wide range of disinfectants needs to be developed, which have a high antimicrobial activity to kill not only the above-mentioned pathogens but also various pathogenic bacteria and viruses causing various diseases, and which can easily disinfect stalls, domestic animals, drinking water, etc.

The term “natural products” refers to materials produced naturally without manipulation by man. In Korea, natural products generally recognized as safe (GRAS) are classified as natural additives and used as food additives without limitations on the amount of use thereof or the kind of food. In foreign countries, they can be freely used as desired by users without special limitations and are used as health foods and medical drugs owing to their excellent functionalities.

Malic acid, a substance abundantly contained in natural fruit, is a substance generally recognized as safe by the United States Food and Drug Administration (US FDA). Malic acid is described as a raw material for medical drugs in notifications provided by the Korean Food and Drug Administration (KFDA) and has been used for a long time as an additive to carbonated drinks, fruits, canned vegetables, fruit juice, candies, chewing gum, etc.

Additionally, grapefruit seed extract contains large amounts of ascorbic acid, naringin and tocopherol, and therefore possesses antimicrobial and antioxidant activities on various foods and inhibits the production of toxic-degradation products. It is a nontoxic, non-metallic, non-corrosive, colorless and odorless natural organic compound, which does not cause environmental contamination and exhibits powerful antibacterial, antiviral and antimold activities against putrefactive and pathogenic microorganisms. When the grapefruit seed extract is absorbed in vivo, it is completely decomposed so that it has no risk of toxicity caused by its accumulation in vivo and thus is safe to humans or animals. Each of malic acid and grapefruit seed extract is used as the raw material of disinfectants and has an acknowledged disinfectant effect. Human beings have inhibited or killed pathogenic bacteria using natural products, such as apricot, peach, plum, garlic and onion, for a long time. Chitosan, which has recently been widely used as an antimicrobial component, is a natural nontoxic biopolymer which is obtained by deacetylating chitin which is the main component of the shell of crabs, shrimps, and other crustaceans. Currently, it is spotlighted while being used for commercial purposes in the biomedical, food and chemical fields. Chitosan and chito-oligomers have a close connection with physiological activities, such as antibacterial and anticancer activities. Chitosan is known to have an excellent ability to inhibit the growth of bacteria compared to chito-oligomers.

In addition, plum has been used for a long time for the prevention and treatment of food poisoning, infectious disease, etc. Currently, it is much studied for the purpose of food preservation, and particularly, contains large amounts of citric acid, malic acid, and related compounds, and correspondingly exhibits a highly excellent bactericidal activity. An extract from plum is known to be effective against all gram-negative and gram-positive bacteria.

At the present time, many efforts to develop disinfectants using natural products are being made worldwide, and commercially available disinfectants include Vircon-S (Bayer Korea) containing malic acid and triple salt as main components, Citruskill (Konipharm International Co., Ltd.) containing grape seed extract and citric acid as main components, and Green-Zone (developed by the present inventors; Korean Patent Registration No. 0528267) containing plum, citric acid and chitosan as main components.

However, considering that the resistance of pathogens becomes stronger with the passage of time due to various factors, such as the long-term use of chemical agents and environmental contamination, natural products should be continuously developed. Accordingly, in the art, there is a need to develop a natural disinfectant which can kill pathogenic bacteria and viruses causing various diseases, has a higher bactericidal activity than that of currently commercially available disinfectants, and can easily disinfect stalls, domestic animals, drinking water, and other areas and materials.

### SUMMARY OF THE INVENTION

The present inventors have made extensive efforts to develop a natural disinfectant having a high bactericidal activity against various pathogenic bacteria and showing the ability to disinfect stalls, as a result, have found that a composition containing malic acid and grapefruit seed extract shows excellent bactericidal and virucidal effects, as compared to compositions containing malic acid or grapefruit seed extract alone. The composition containing malic acid and grapefruit seed extract has been named “Green-zone A” and is sometimes hereinafter referred to by such name.

The present invention provides a bactericidal and virucidal composition containing malic acid and grapefruit seed extract.

In one preferred embodiment, the composition according to the present invention may additionally contain peppermint oil. In other preferred embodiments, the composition may additionally contain at least one ingredient selected from among plum extract, citric acid and chitosan.

In one embodiment of the inventive composition, the grapefruit seed extract is contained in an amount of 1-30 parts by weight, based on 100 parts by weight of the malic acid.

The bactericidal and virucidal action of compositions of the invention are advantageously utilized in a wide variety of disinfection applications involving a broad spectrum of bacterial and viral species. For example, bacteria may include at least one of Salmonella cholerasuis, Salmonella typhimurium, Salmonella enteritidis, Staphylococcus aureus, Streptococcus suis, L. monocytogenes, E. coli and V. parahaemolyticus, and viruses may include at least one of avian influenza virus, Foot-and-mouth disease virus, hog cholera virus, transmissible gastroenteritis virus, canine parvovirus and canine distemper virus.

In one aspect, the invention relates to a bactericidal and virucidal composition, including malic acid and grapefruit seed extract, wherein the grapefruit seed extract is present in the composition in an amount of 1-30 parts by weight, based on 100 parts by weight of the malic acid, and at least one additional ingredient selected from among peppermint oil, plum extract, citric acid and chitosan.

In another aspect, the invention relates to a method of disinfecting a locus susceptible to presence of bacterial and/or viral infection, including administering to such locus an effective amount of a composition of the present invention.

A still further aspect of the invention relates to a method of disinfecting drinking water susceptible to presence of bacterial and/or viral infection, comprising treating said drinking water with an effective amount of a composition of the present invention.

The above and other aspects, objects, features and embodiments of the present invention will be more clearly understood from the following detailed description and the accompanying claims.

### DETAILED DESCRIPTION OF THE INVENTION, AND PREFERRED EMBODIMENTS THEREOF

The malic acid which is used as one of essential components in the composition according to the present invention is widely known to have bactericidal and virucidal effects and is generally recognized as safe (GRAS) by US FDA. DL-malic acid has a chemical formula of C₄H₆O₅, a molecular weight of 134.09, a specific gravity of 1.601, a melting point of 133° C., and a boiling point of 150° C. (decomposition). DL-malic acid has no deliquescence, is about 20% higher in acidity than citric acid and is abundantly contained in natural fruits, such as apple, grape and the like. When the acidic malic acid is introduced into microorganisms, the microorganisms draw in organic acids to maintain pH homeostasis. In this mechanism, the microbial cytoplasm will be acidified and will affect enzymatic reactions and mass transfer processes and will consume energy for pH homeostasis and thus the microorganisms will be killed.

The grapefruit seed extract, which is another essential component in the inventive composition, is listed in the US FDA food additive list, and has passed the toxicity test. Also, it has nontoxic and non-metallic characteristics, and thus has no limitations on the amount of use thereof and foods to which it can be added. It typically contains about 4.5% of ascorbic acid and is rich in ascorbic acid, palmitic acid, ester, tocopherol, etc., and thus has powerful bactericidal activities, tonic effects, neutralizing effects against poisonous matter, and the effect of enhancing the antimicrobial activity of leucocytes. In addition, it has the effect of preventing the auto-oxidation of oil and fat, which can occur during the storage of food, as well as the effect of inhibiting the production of toxic substances caused by the oxidation.

The peppermint oil, which can be employed as another component in compositions of the invention, is an essential oil obtained by distilling the leaf or stem of peppermint with steam. Peppermint is grown naturally or cultivated in Asia, Europe and North America. The main components of peppermint oil are menthol and menthone, and peppermint oil also contains menthyl ester, pinen, limonene, etc. Peppermint oil is colorless or light-yellowish liquid, has peculiar fragrance and strong hot taste, and is soluble in alcohol. It is used as a perfume ingredient in dental paste, toothpaste, cosmetics, confectionary, liquors, and medical drugs.

### EXAMPLES

Hereinafter, the present invention will be more fully described in detail by examples. It is to be understood, however, that these examples are for illustrative purpose only and are not intended to be construed to limit the scope of the present invention.

### Example 1

### Preparation of Antimicrobial Composition

1-1: Preparation of Green-Zone A

50 g of malic acid (Yongsan Chemicals, Inc., Korea) was completely dissolved in 200 ml of purified water to prepare solution 1, and 6 g of grapefruit seed extract (AF Bank, Korea) was completely dissolved in 200 ml of purified water to prepare solution 2. 5-10 ml of each of the solutions 1 and 2 and peppermint oil (Bolak Ltd, Korea) was put in a mixing tank into which purified water was then charged to a final volume of 1 liter. The resulting solution was thoroughly mixed at 2,000 rpm for 2 minutes. The mixed solution was filtered through a 5 μm filter, thus preparing antimicrobial composition “Green-zone A”.

1-2: Preparation of Green-Zone B

30 g of malic acid (Yongsan Chemicals, Inc., Korea) was completely dissolved in 120 ml of purified water to prepare solution 3, and 3 g of grapefruit seed extract (AF Bank, Korea), 10 g of plum concentrate (MSC, Korea), 10 g of citric acid (Trion, Korea) and 3 g of chitosan (Chitolife, Korea) were completely dissolved in 120 ml of distilled water to prepare solution 4. 5-10 ml of each of the solutions 3 and 4 and peppermint oil (Bolak, Korea) was put in a mixing tank into which purified water was then charged to a final volume of 1 liter. The resulting solution was thoroughly mixed at 2,000 rpm for 2 minutes. The mixed solution was filtered through a 5 μm filter, thus preparing antimicrobial composition “Green-zone B”.

### Example 2

### Antibacterial Effects

2-1: Bactericidal Effects Against Salmonella Strains

Bacterial strains used in antimicrobial tests in this Example were Salmonella cholerasuis, Salmonella typhimurium, Salmonella enteritidis, Streptococcus suis, Staphylococcus aureus, Listeria monocytogenes, E. coli and Vibrio parahaemolyticus. The test strains were cultured in nutrient media at 37° C. for 24 hours, and used at a cell concentration of 5×10⁸ cells/ml of the nutrient media.

Green-zone A prepared in Example 1 was undiluted and diluted 2-fold, 5-fold, 10-fold and 20-fold with hard water. Each of the solutions was divided into groups 1 through 5, in which the group 1 was treated with distilled water to have no organic matter, the group 2 was treated with hard water to have a low organic matter content, the group 3 was treated with 1% FBS and 5% FBS to have high organic matter content, the group 4 was used as a control to the groups 2 and 3, and the group 5 was used as a control to the group 1.

4 ml of each of the prepared bacterial strains was mixed with 96 ml of 5% organic matter dilution (4° C., 5% yeast extract, pH 7.0, diluted with hard water). 2.5 ml of the mixture was taken and allowed to react at 4° C. for 30 minutes. After completion of the reaction, 1 ml of each of the bacterial dilutions was mixed with 9 ml of neutralizing medium (containing 5% horse serum inactivated in nutrient medium) at 37° C., and added into each of five test tubes containing 0.1 ml of each of nutrient media, and then cultured at 37° C. for 48 hours.

After completion of the proliferation, each of the test bacterial strains was re-inoculated into blood medium and measured for bactericidal activity (Tables 1-8). As a result, as shown in Table 1, Green-zone A according to the present invention showed disinfectant effects on Salmonella cholerasuis up to dilutions of 1:2 for all of the groups 1 through 3. Also, as shown in Table 2, Green-zone A showed disinfectant effects on Salmonella typhimurium up to dilutions of 1:10. Also, as shown in Table 3, Green-zone A showed disinfectant effects on Salmonella enteritidis up to dilutions of 1:20.

As shown in Table 4, Green-zone A according to the present invention showed disinfectant effects on Streptococcus suis up to dilutions of 1:20 for all the groups 1 through 3. As shown in Table 5, Green-zone A according to the present invention showed disinfectant effects on Staphylococcus aureus up to dilutions of 1:2, and as shown in Table 6, it showed disinfectant effects on Listeria monocytogenes up to dilutions of 1:20.

As shown in Table 7, Green-zone A according to the present invention showed disinfectant effects on E. coli up to dilutions of 1:5 for all of the groups 1 through 3. As shown in Table 8, Green-zone A according to the present invention showed disinfectant effects on Vibrio parahaemolyticus up to dilutions of 1:10.

Accordingly, it was found that Green-zone A according to the present invention has highly excellent antibacterial effects on a broad spectrum of bacteria.

TABLE 1

| Sal. | | | Group 3 | | | |
|---|---|---|---|---|---|---|
| cholerasuis | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | BD | BD | BD |
| solution of | | | | | | |
| Green-zone A | | | | | | |

NBD: No Bacteria Detected; BD: Bacteria Detected; FBS: Fetal Bovine Serum

TABLE 2

| Sal. | | | Group 3 | | | |
|---|---|---|---|---|---|---|
| typhimurium | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | NBD | NBD | BD | BD |
| solution of | | | | | | |
| Green-zone A | | | | | | |
| 1:20 diluted | NBD | NBD | BD | BD | BD | BD |
| solution of | | | | | | |
| Green-zone A | | | | | | |

TABLE 3

| | | | Group 3 | | | |
|---|---|---|---|---|---|---|
| Sal. enteritidis | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | NBD | NBD | BD | BD |
| solution of | | | | | | |
| Green-zone A | | | | | | |
| 1:20 diluted | NBD | NBD | NBD | NBD | BD | BD |
| solution of | | | | | | |
| Green-zone A | | | | | | |
| 1:60 diluted | BD | BD | BD | BD | BD | BD |
| solution of | | | | | | |
| Green-zone A | | | | | | |

TABLE 4

| | | | Group 3 | | | |
|---|---|---|---|---|---|---|
| Stp. suis | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | NBD | NBD | BD | BD |
| solution of | | | | | | |
| Green-zone A | | | | | | |
| 1:20 diluted | NBD | NBD | NBD | NBD | BD | BD |
| solution of | | | | | | |
| Green-zone A | | | | | | |
| 1:60 diluted | BD | BD | BD | BD | BD | BD |
| solution of | | | | | | |
| Green-zone A | | | | | | |

TABLE 5

| | | | Group 3 | | | |
|---|---|---|---|---|---|---|
| Sta. aureus | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | BD | BD | BD | BD | BD | BD |
| solution of | | | | | | |
| Green-zone A | | | | | | |

TABLE 6

| L. | | | Group 3 | | | |
|---|---|---|---|---|---|---|
| monocytogenes | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | NBD | NBD | BD | BD |
| solution of | | | | | | |
| Green-zone A | | | | | | |
| 1:20 diluted | NBD | NBD | NBD | NBD | BD | BD |
| solution of | | | | | | |
| Green-zone A | | | | | | |
| 1:60 diluted | NBD | BD | BD | BD | BD | BD |
| solution of | | | | | | |
| Green-zone A | | | | | | |

TABLE 7

| | | | Group 3 | | | |
|---|---|---|---|---|---|---|
| E. coli | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | BD | BD | BD | BD |
| solution of | | | | | | |
| Green-zone A | | | | | | |

TABLE 8

| V. para- | | | Group 3 | | | |
|---|---|---|---|---|---|---|
| haemolyticus | Group 1 | Group 2 | 1% FBS | 5% FBS | Group 4 | Group 5 |
| Green-zone A | NBD | NBD | NBD | NBD | BD | BD |
| 1:2 diluted | NBD | NBD | NBD | NBD | BD | BD |
| solution of | | | | | | |
| Green-zone A | | | | | | |
| 1:5 diluted | NBD | NBD | NBD | NBD | BD | BD |
| solution of | | | | | | |
| Green-zone A | | | | | | |
| 1:10 diluted | NBD | NBD | NBD | NBD | BD | BD |
| solution of | | | | | | |
| Green-zone A | | | | | | |
| 1:20 diluted | BD | BD | BD | BD | BD | BD |
| solution of | | | | | | |
| Green-zone A | | | | | | |

### Example 3

### Antiviral Effects

3-1: Virucidal Effects on AI Virus

For virucidal test, as avian influenza viruses (AIV; field isolate strain No. 02-8; virus strain K228; Ichon, Kyunggi-Do, 2002), active viruses which have been in subculture were used at a concentration of more than 10⁷/ml.

Green-zone A prepared in Example 1 was undiluted and diluted 2-fold and 5-fold with hard water. Each of the solutions was divided into groups 1 through 5, in which the group 1 was treated with distilled water to have no organic matter, the group 2 was treated with hard water to have a low organic matter content, the group 3 was treated with 1% FBS and 5% FBS to have high organic matter contents, the group 4 was used as a control to the groups 2 and 3, and the group 5 was used as a control to the group 1.

1 ml of the viral solution (4° C.) was mixed with 24 ml of an organic matter solution for dilution (4° C., hard water containing FBS). 2.5 ml of the viral mixture solution was put in each of test tubes containing the same amount (2.5 ml) of the Green-zone A dilution (4° C.) to make a total volume of 5 ml, and then allowed to react at 4° C. for 30 minutes.

After completion of the reaction, to neutralize the efficacy of the disinfectant, 5 ml of neutralizing solution (37° C., PBS containing 50% FBS) was added and mixed with the reaction solution. The neutralized solution was undiluted and diluted 10⁻¹, 10⁻², 10⁻³, 10⁻⁴ and 10⁻⁵ fold with PBS, and 0.2 ml of each of the diluted solutions was inoculated into the allantoic cavity of five 10-day-old eggs.

Then, the inoculated eggs were incubated for 5 days, and the allantoic fluid was collected and subjected to a hemagglutination reaction using 10% chicken red blood cells to examine the presence or absence of viruses and viral titer (Table 9). As a result, as shown in Table 9, it was found that Green-zone A according to the present invention showed disinfectant effects up to dilutions of 1:2 for all of the groups 1 through 3, indicating virucidal effects.

TABLE 9

| | | | Group 3 | | | |
|---|---|---|---|---|---|---|
| S. cholerasuis | Group 1 | Group 2 | 1% FBS | 5% FBS | Group 4 | Group 5 |
| Green-zone A | NVD | NVD | NVD | NVD | VD | VD |
| 1:2 diluted | NVD | NVD | NVD | NVD | VD | VD |
| solution of | | | | | | |
| Green-zone A | | | | | | |
| 1:5 diluted | NVD | NVD | VD | VD | VD | VD |
| solution of | | | | | | |
| Green-zone A | | | | | | |

NVD: No Virus Detected; VD: Virus Detected

3-2: Virucidal Effects on Foot-and-Mouth Virus

To measure the virucidal effects of Green-zone A according to the present invention, Green-zone A was tested for efficacy in the Institute for Animal Health (Pirbright Laboratory) designated as the World Reference Laboratory for Foot-and-Mouth Disease by the Office International des Epizootics (OIE).

First, BHK21 cells were cultured. The cultured cells were inoculated with foot-and-mouth viruses which have been brought into contact with 4-fold, 6-fold, 8-fold and 10-fold dilutions of the Green-zone A disinfectant of the present invention at 4° C. for 30 minutes, and the inoculated cells were cultured at 37° C. for 48 hours. After completion of the culture, the cells were stained with methylene blue and examined for the presence of plaques under natural light (Table 10). As a result, as shown in 10, it was found that Green-zone A according to the present invention kills foot-and-mouth viruses up to dilutions of 1:10, even in the presence of organic matter.

TABLE 10

| Dilution fold | 1% fetal serum | pH |
|---|---|---|
| 1:4 | NVD | 3.14 |
| 1:6 | NVD | 3.15 |
| 1:8 | NVD | 3.15 |
| 1:10 | NVD | 3.41 |

3-3: Virucidal Effects on Hog Cholera Virus and Transmissible Gastroenteritis Virus

For virucidal tests, as hog cholera virus (HCV, LOM strain) and transmissible gastroenteritis virus (TGEV, strain Pyungtak-40), active viruses which have been in subculture were used at a concentration of more than 10^{4.5}/ml (TCID₅₀/0.1ml).

Green-zone A prepared in Example 1 was undiluted and diluted 2-fold, 5-fold and 10-fold with hard water. Each of the solutions was divided into groups 1 through 5, in which the group 1 was treated with distilled water to have no organic matter, the group 2 was treated with hard water to have a low organic matter content, the group 3 was treated with 1% FBS and 5% FBS to have high organic matter contents, the group 4 was used as a control to the groups 2 and 3, and the group 5 was used as a control to the group 1.

1 ml of the viral solution (4° C.) was mixed with 19.0 ml of an organic matter solution for dilution (4° C., hard water containing FBS). 2.5 ml of the mixture solution was put in each of test tubes containing the same amount (2.5 ml) of each of the Green-zone A dilutions (4° C.) to make a total volume of 5 ml and then allowed to react at 4° C. for 30 minutes.

After completion of the reaction, to neutralize the efficacy of the disinfectant (Green-zone A), the same amount (5 ml) of neutralizing solution (PBS containing 50% FBS, 37° C.) was added and mixed with each of the reaction solutions. Each of the neutralized solutions was undiluted and diluted 10⁻¹, 10⁻², 10⁻³, 10⁻⁴ and 10⁻⁵ fold with PBS, and each of the dilutions was inoculated into the cells and calculated for viral content.

In inoculation with the disinfectant, a medium was removed from a plate where PK-15 cells had been monolayer-cultured, and the plate was washed with PBS. 100 μl of each of the disinfectant dilutions was inoculated four times into each well of the plate. As a negative control group, 200 μl of medium was added. After the inoculation, adsorption was conducted for 30 minutes, and the medium was replaced. The plate was incubated in an CO₂ incubator at 37° C. for 72 hours and examined for the proliferation of viruses using the IDEXX classical swine fever virus detection kit (99-43300), in which an optical density of more than 0.300 was evaluated as positive (Tables 11 and 12). As a result, as shown in Tables 11 and 12, Green-zone A according to the present invention showed disinfectant effects up to dilutions of 1:5 for all of the groups 1 through 3, indicating virucidal effects.

TABLE 11

| Hog cholera | | | Group 3 | | | |
|---|---|---|---|---|---|---|
| virus | Group 1 | Group 2 | 1% FBS | 5% FBS | Group 4 | Group 5 |
| Green-zone A | NVD | NVD | NVD | NVD | VD | VD |
| 1:2 diluted | NVD | NVD | NVD | NVD | VD | VD |
| solution of | | | | | | |
| Green-zone A | | | | | | |
| 1:5 diluted | NVD | NVD | NVD | NVD | VD | VD |
| solution of | | | | | | |
| Green-zone A | | | | | | |
| 1:10 diluted | VD | VD | VD | VD | VD | VD |
| solution of | | | | | | |
| Green-zone A | | | | | | |

TABLE 12

| | | | Group 3 | | | |
|---|---|---|---|---|---|---|
| TGEV | Group 1 | Group 2 | 1% FBS | 5% FBS | Group 4 | Group 5 |
| Green-zone A | NVD | NVD | NVD | NVD | VD | VD |
| 1:2 diluted | NVD | NVD | NVD | NVD | VD | VD |
| solution of | | | | | | |
| Green-zone A | | | | | | |
| 1:5 diluted | NVD | NVD | NVD | NVD | VD | VD |
| solution of | | | | | | |
| Green-zone A | | | | | | |
| 1:10 diluted | VD | VD | VD | VD | VD | VD |
| solution of | | | | | | |
| Green-zone A | | | | | | |

3-4: Virucidal Effects on Canine Parvovirus and Canine Distemper Virus

For virucidal tests, as canine parvovirus (CPV, C-780916 strain) and canine distemper virus (CDV, Lederle strain), active viruses which have been in subcultutre were used at a concentration of 10^{4.0}/ml (TCID₅₀/ml).

Green-zone A prepared in Example 1 was undiluted and diluted 2-fold, 5-fold and 10-fold with hard water. Each of the solutions was divided into groups 1 through 5, in which the group 1 was treated with distilled water to have no organic matter, the group 2 was treated with hard water to have a low organic matter content, the group 3 was treated with 1% FBS and 5% FBS to have high organic matter contents, the group 4 was used as a control to the groups 2 and 3, and the group 5 was used as a control to the group 1.

1 ml of the viral solution (4° C.) was mixed with 19.0 ml of an organic matter solution for dilution (4° C., hard water containing FBS). 2.5 ml of the viral mixture solution was put in each of test tubes containing the same amount (2.5 ml) of the Green-zone A dilution (4° C.) to make a total volume of 5 ml, and then allowed to react at 4° C. for 30 minutes.

After completion of the reaction, to neutralize the efficacy of the disinfectant (Green-zone A), the same amount (5 ml) of neutralizing solution (PBS containing 50% FBS, 37° C.) was added and mixed with each of the reaction solutions. Each of the neutralized solutions was undiluted and diluted 10⁻¹, 10⁻², 10⁻³, 10⁻⁴ and 10⁻⁵ fold with PBS, and each of the dilutions was inoculated into the cells and calculated for viral content.

A medium was removed from a 96-well plate where cells have been monolayer cultured, and the plate was washed with PBS, and 4 wells of the plate were inoculated with each of the disinfectant dilutions. The cell lines used were CRFK cells (Crandell feline kidney cell) for CPV and Vero cells for CDV. The cells to be inoculated with CPV were plated 9-10 hours before inoculation.

The inoculated plate was incubated for 2-3 days and subjected to a freezing and thawing process 3 times. 50 μl of each of the cell lines in the first plate was added into another 96-well plate to which 50 μl of 0.7% cold pig RBC was added. After 1 hour, reading was performed.

Specifically, a medium was removed from a plate where cells to be inoculated with the disinfectant had been monolayer cultured, and the plate was washed with PBS. Each well of the plate was inoculated four times with 100 μl of each of the disinfectant dilutions, and as a negative control group, 200 μl of medium was added. Cell lines used in this Example were CRFK cells (Crandell feline kidney cell) for CPV and Vero cells for CDV. The cells to be inoculated with CPV were plated 9-10 hours before inoculation. After inoculation, adsorption was conducted for 30 minutes and then the medium was replaced. Then, the plate was incubated in a CO₂ incubator at 37° C. for 72 hours, and 50 μl of 0.7% cold pig RBC was added thereto. After 1 hour, reading was performed (Tables 13 and 14). As a result, Green-zone A according to the present invention showed disinfectant effects up to dilutions of 1:5, indicating virucidal effects.

TABLE 13

| Canine Parvo | | | | | | |
|---|---|---|---|---|---|---|
| Virus C- | | | Group 3 | | | |
| 780916 strain | Group 1 | Group 2 | 1% FBS | 5% FBS | Group 4 | Group 5 |
| Green-zone A | NVD | NVD | NVD | NVD | VD | VD |
| 1:2 diluted | NVD | NVD | NVD | NVD | VD | VD |
| solution of | | | | | | |
| Green-zone A | | | | | | |
| 1:5 diluted | NVD | NVD | NVD | NVD | VD | VD |
| solution of | | | | | | |
| Green-zone A | | | | | | |
| 1:10 diluted | NVD | NVD | VD | VD | VD | VD |
| solution of | | | | | | |
| Green-zone A | | | | | | |

TABLE 14

| Canine Dis- | | | | | | |
|---|---|---|---|---|---|---|
| temper Virus | | | Group 3 | | | |
| Lederle strain | Group 1 | Group 2 | 1% FBS | 5% FBS | Group 4 | Group 5 |
| Green-zone A | NVD | NVD | NVD | NVD | VD | VD |
| 1:2 diluted | NVD | NVD | NVD | NVD | VD | VD |
| solution of | | | | | | |
| Green-zone A | | | | | | |
| 1:5 diluted | NVD | NVD | NVD | NVD | VD | VD |
| solution of | | | | | | |
| Green-zone A | | | | | | |
| 1:10 diluted | NVD | NVD | NVD | NVD | VD | VD |
| solution of | | | | | | |
| Green-zone A | | | | | | |
| 1:20 diluted | NVD | NVD | VD | VD | VD | VD |
| solution of | | | | | | |
| Green-zone A | | | | | | |

### Example 4

### Comparison of Effects Between Green-Zone A and Each of Malic Acid and Grapefruit Seed Extract

Comparison of Antibacterial Effects

The antibacterial effect of Green-zone A prepared in Example 1 was compared to the effect of each of malic acid and grapefruit seed extract alone.

A bacterial strain used in the test of antibacterial activity was Salmonella cholerasuis. The Salmonella strain was cultured in nutrient medium at 37° C. for 24 hours and used at a concentration of 5×10⁸ cells/ml of the nutrient medium.

Green-zone A prepared in Example 1 was undiluted and diluted 2-fold and 5-fold with hard water. Meanwhile, a composition of 50 g malic acid completely dissolved in 200 ml of purified water, and a composition of 6 g grapefruit seed extract completely dissolved in 200 ml of distilled water, were undiluted and diluted 2-fold and 5-fold.

4 ml of the prepared Salmonella strain was mixed with 96 ml of 5% organic matter dilution (4° C., 5% yeast extract, pH 7.0, diluted with hard water). 2.5 ml of the mixture solution was taken and allowed to react at 4° C. for 30 minutes.

After completion of the reaction, 1 ml of the bacterial dilution was added and mixed with 9 ml of neutralizing medium (containing 5% horse serum inactivated in nutrient medium) at 37° C. Then, the mixture was added into test tubes containing 0.1 ml of each of nutrient media, and was proliferated in an incubator at 37° C. for 48 hours.

After completion of the proliferation, the viral solution was re-inoculated into blood medium and measured for the bactericidal activity of each of the compositions (Table 15). As a result, as shown in Table 15, Green-zone A according to the present invention had disinfectant effects up to dilutions of 1:2, but each of the malic acid and the grapefruit seed extract had disinfectant effects up to dilutions of 1:1. This suggests that Green-zone A according to the present invention has excellent disinfectant effects, as compared to each of malic acid and grapefruit seed extract alone.

TABLE 15

| Dilution fold | Malic acid | Grapefruit seed extract | Green-zone A |
|---|---|---|---|
| Original solution | NBD | NBD | NBD |
| 1:2 | BD | BD | NBD |
| 1:5 | BD | BD | BD |

4-2: Comparison of Virucidal Effects

For virucidal tests, as avian influenza viruses (AIV) (field isolate No. 02-8, virus strain K228; Ichon, Kyunggi-Do, 2002), active viruses which have been in subculture were used at a concentration of more than 10⁷/ml.

Green-zone A prepared in Example 1 was undiluted and diluted 2-fold and 5-fold with hard water. Meanwhile, a composition of 50 g malic acid completely dissolved in 200 ml of purified water, and a composition of 6 g grapefruit seed extract completely dissolved in 200 ml of purified water, were undiluted and diluted 2 and 5 fold.

1 ml of the viral solution was mixed with 24 ml of an organic matter solution for dilution (4° C., hard water containing FBS). 2.5 ml of the viral mixture solution was added into test tubes each containing the same amount (2.5 ml) of the disinfectant solution (4° C.) to make a total volume of 5 ml, and then allowed to react at 4° C. for 30 minutes.

After completion of the reaction, to neutralize the efficacy of the disinfectant, the same amount (5 ml) of neutralizing solution (37° C., PBS containing 50% FBS) was added and mixed with the reaction solution. The neutralized solution was undiluted and diluted 10⁻¹, 10⁻², 10⁻³, 10⁻⁴ and 10⁻⁵ fold with PBS, and 0.2 ml of each of the diluted solutions was inoculated into the allantoic cavity of five 10-day-old eggs.

Then, the inoculated eggs were incubated for 5 days, and the allantoic fluid was collected and subjected to a hemagglutination reaction using 10% chicken red blood cells to examine the presence or absence of viruses and viral titer (Table 16). As a result, as shown in Table 16, Green-zone A according to the present invention had disinfectant effects up to dilutions of 1:2, but the malic acid had no disinfectant effects and the grapefruit seed extract had disinfectant effects in the undiluted solution. This indicates that Green-zone A according to the present invention has excellent disinfectant effects, as compared to each of malic acid and grapefruit seed extract alone.

TABLE 16

| Dilution fold | Malic acid | Grapefruit seed extract | Green-zone A |
|---|---|---|---|
| Original solution | VD | NVD | NVD |
| 1:2 | VD | VD | NVD |
| 1:5 | VD | VD | NVD |

NVD: No Virus Detected/VD: Virus Detected

### Example 5

### Comparison of Effects Between Green-Zone A and Green-Zone B

5-1: Comparison of Antibacterial Effects

The antibacterial effect of Green-zone A prepared in Example 1 was compared to the antibacterial effect of Green-zone B.

A bacterial strain used in the test of antibacterial activity was Salmonella cholerasuis. The Salmonella strain was cultured in nutrient medium at 37° C. for 24 hours and used at a concentration of 5×10⁸ cells/ml of the nutrient medium. Green-zone A and Green-zone B prepared in Example 1 were undiluted and diluted 2-fold, 5-fold and 10-fold with hard water.

4 ml of the prepared Salmonella strain was mixed with 96 ml of 5% organic matter dilution (4° C., 5% yeast extract, pH 7.0, diluted with hard water). 2.5 ml of the mixture solution was taken and allowed to react at 4° C. for 30 minutes.

After completion of the reaction, 1 ml of the bacterial dilution was added and mixed with 9 ml of neutralizing medium (containing 5% horse serum inactivated in nutrient medium) at 37° C. Then, the mixture was added into test tubes containing 0.1 ml of each of nutrient media, and was proliferated in an incubator at 37° C. for 48 hours.

After completion of the proliferation, the viral solution was re-inoculated into blood medium and measured for the bactericidal activity of each of the compositions (Table 17). As a result, as shown in Table 17, Green-zone A according to the present invention had disinfectant effects up to dilutions of 1:2, and Green-zone B had disinfectant effects up to dilutions of 1:5. This indicates that Green-zone B has excellent antibacterial effects, as compared to Green-zone A.

TABLE 17

| Dilution fold | Green-zone A | Green-zone B |
|---|---|---|
| Original solution | NBD | NBD |
| 1:2 | NBD | NBD |
| 1:5 | BD | NBD |
| 1:10 | BD | BD |

5-2: Comparison of Virucidal Effects Between Green-Zone A and Green-Zone B

For virucidal tests, as avian influenza viruses (AIV) (field isolate No. 02-8, virus strain K228; Ichon, Kyunggi-Do, 2002), active viruses which have been in subculture were used at a concentration of more than 10⁷/ml. Green-zone A and Green-zone B prepared in Example 1 were undiluted and diluted 2-fold, 5-fold and 10-fold with hard water.

1 ml of the viral solution (4° C.) was mixed with 24 ml of an organic matter solution for dilution (4° C., hard water containing FBS). 2.5 ml of the viral mixture solution was added into test tubes containing the same amount (2.5 ml) of each of the disinfectant solutions (4° C.) to make a total volume of 5 ml, and then allowed to react at 4° C. for 30 minutes.

After completion of the reaction, to neutralize the efficacy of the disinfectants, the same amount (5 ml) of neutralizing solution (37° C., PBS containing 50% FBS) was added and mixed with each of the reaction solutions. The neutralized solution was undiluted and diluted 10⁻¹, 10⁻², 10⁻³, 10⁻⁴ and 10⁻⁵ fold with PBS, and 0.2 ml of each of the diluted solutions was inoculated into the allantoic cavity of five 10-day-old eggs.

Then, the inoculated eggs were incubated for 5 days, and the allantoic fluid was collected and subjected to a hemagglutination reaction using 10% chicken red blood cells to examine the presence or absence of viruses and viral titer (Table 18). As a result, as shown in Table 18, Green-zone A according to the present invention had disinfectant effects up to dilutions of 1:2, and Green-zone B had disinfectant effects up to dilutions of 1:5. This indicates that Green-zone B has excellent virucidal effects, as compared to Green-zone A.

TABLE 18

| Dilution fold | Green-zone A | Green-zone B |
|---|---|---|
| Original solution | NVD | NVD |
| 1:2 | NVD | NVD |
| 1:5 | VD | NVD |
| 1:10 | VD | VD |

As described above in detail, the disinfectant composition according to the present invention is made of natural products and shows disinfectant effects on a broad spectrum of bacteria and viruses even with high organic matter content, and thus is particularly useful for the disinfection of stalls, domestic animals and drinking water.

Although specific embodiments of the present invention have been described in detail above, those skilled in the art will appreciate that these descriptions are only intended to give preferred embodiments and are not intended to be construed to limit the scope of the present invention. Accordingly, the substantial scope of the present invention will be defined by claims and equivalents thereof.

What is claimed is:

1. A bactericidal and virucidal composition containing malic acid and grapefruit seed extract.

2. The bactericidal and virucidal composition according to claim 1, which additionally contains peppermint oil.

3. The bactericidal and virucidal composition according to claim 1, which additionally contains at least one ingredient selected from among plum extract, citric acid and chitosan.

4. The bactericidal and virucidal composition according to claim 1, wherein the grapefruit seed extract is present in the composition in an amount of 1-30 parts by weight, based on 100 parts by weight of the malic acid.

5. The bactericidal and virucidal composition according to claim 1, having cidal activity against at least one bacterium selected from among Salmonella cholerasuis, Salmonella typhimurium, Salmonella enteritidis, Staphylococcus aureus, Streptococcus suis, L. monocytogenes, E. coli and V. parahaemolyticus.

6. The bactericidal and virucidal composition according to claim 1, having cidal activity against at least one virus selected from among avian influenza virus, Foot-and-mouth disease virus, hog cholera virus, transmissible gastroenteritis virus, canine parvovirus and canine distemper virus.

7. A bactericidal and virucidal composition, comprising malic acid and grapefruit seed extract, wherein the grapefruit seed extract is present in the composition in an amount of 1-30 parts by weight, based on 100 parts by weight of the malic acid, and at least one additional ingredient selected from among peppermint oil, plum extract, citric acid and chitosan.

8. A method of disinfecting a locus susceptible to presence of bacterial and/or viral infection, comprising administering to such locus an effective amount of a composition according to claim 1.

9. The method of claim 8, wherein the locus is selected from among stalls, domestic animals and drinking water.

10. The method of claim 8, wherein said bacterial and/or viral infection comprises presence in said locus of at least one bacterial and/or viral species selected from among Salmonella cholerasuis, Salmonella typhimurium, Salmonella enteritidis, Staphylococcus aureus, Streptococcus suis, L. monocytogenes, E. coli, V. parahaemolyticus, avian influenza virus, Foot-and-mouth disease virus, hog cholera virus, transmissible gastroenteritis virus, canine parvovirus and canine distemper virus.

11. A method of disinfecting drinking water susceptible to presence of bacterial and/or viral infection, comprising treating said drinking water with an effective amount of a composition according to claim 1.

12. The method of claim 11, wherein said bacterial and/or viral infection comprises presence in said drinking water of at least one bacterial and/or viral species selected from among Salmonella cholerasuis, Salmonella typhimurium, Salmonella enteritidis, Staphylococcus aureus, Streptococcus suis, L. monocytogenes, E. coli, V. parahaemolyticus, avian influenza virus, Foot-and-mouth disease virus, hog cholera virus, transmissible gastroenteritis virus, canine parvovirus and canine distemper virus.
